(19) United States
(12) Patent Application Publication
(10) Pub. No.: US 2012/0195835 A1
(43) Pub. Date: Aug. 2, 2012

(54) TUMOR TISSUE-SELECTIVE BIO-IMAGING NANOPARTICLES

(75) Inventors: Kyoungja WOO, Seoul (KR); Myung-Ik YOO, Seoul (KR)

(73) Assignee: KOREA INSTITUTE OF SCIENCE AND TECHNOLOGY, Seoul (KR)

(21) Appl. No.: 13/359,870

(22) Filed: Jan. 27, 2012

(30) Foreign Application Priority Data
Jan. 28, 2011 (KR) 10-2011-0009110

(51) Int. Cl. A61K 49/10; B82Y 15/00

(52) U.S. Cl. 424/9.32; 977/810; 977/930

(57) ABSTRACT

A bio-imaging nanoparticle is composed of a core nanoparticle, a bonding layer having organic ligands, surfactants and polyoxyalkylene derivatives of fatty acid ester, and veiling the core nanoparticle, and functional molecules, wherein the organic ligands are bound to a surface of the core nanoparticle, the surfactants are bound to a portion of the surface of the core nanoparticle to which the organic ligands are not bound, the polyoxyalkylene derivatives of the fatty acid ester are introduced in an empty space between the organic ligands and the surfactants of the bonding layer, and the functional molecule is bound to a second terminal end opposite to a first terminal end of both terminal ends of the organic ligand, the first terminal end of the organic ligand being bound to a shell of the core nanoparticle.

### CROSS-REFERENCE TO RELATED APPLICATION

The present disclosure relates to subject matter contained in priority Korean Application No. 10-2011-0009110, filed on Jan. 28, 2011, which is herein expressly incorporated by reference in its entirety.

### BACKGROUND OF THE INVENTION

1. Field of the Invention

This specification relates to bio-imaging (contrast agents) nanoparticles generally used for disease diagnosis, and particularly, to bio-imaging (contrast agents) nanoparticles with improved targetability and tumor tissue-selectivity.

2. Background of the Invention

In recent time, MR contrast agents nanoparticles, such as Ferridex or Resovist, which is produced by coating dextran on superparamagnetic iron oxide nanoparticles, are used to diagnose diseases associated with liver tissues. Since such contrast agents are produced with the dextran polymer coated on iron oxide nanoparticles less than 10 nm in size, many numbers of nanoparticles are coated together in view of a characteristic of the polymer, by which the nanoparticles are unable to be individually dispersed on water but aggregated into a group. Hence, upon measuring a hydrodynamic size, it is approximately 160 nm and 58 nm, and a TEM image exhibits a shape of cluster of particles. The size of the particles allows the particles to be seized by an immune system when being injected into a body so as to be delivered to the liver and the spleen. Thus, those particles are used as the liver contrast agents. When the hydrodynamic size of the nanoparticles is extremely small less than 7 nm, the nanoparticles are likely to flow out of blood vessels via a gap between endothelial cells. Therefore, in order to effectively transfer superparamagnetic iron oxide nanoparticles to lesions other than the liver tissues to realize a diagnosis by a contrast effect, it is preferable to use iron oxide nanoparticles having the hydrodynamic size slightly smaller than about 10 nm. The characteristic of the contrast agent effect corresponds to most of other bio-imaging nanoparticles other than the superparamagnetic iron oxide nanoparticles.

Also, in addition to the 10-nm hydrodynamic size, the nanoparticles should have a bond to molecules exhibiting target selectivities (targetability) for a specific marker which is more frequently expressed only in lesions, and exhibit excellent biocompatibility and superior dispersibility in water (water-solubility) so as to be used as injection.

As a nanoparticle synthesis technology and a surface modification technology are developed, many efforts are being made to develop contrast agents satisfying those characteristics, but even now, the respective quantities of nanoparticles delivered to the liver and the spleen are several times and ten times larger than that delivered to a specific lesion. This is because most of the nanoparticles are successfully coupled to dispersible (water-soluble) molecules, biocompatible molecules and targeting molecules, but the hydrodynamic size thereof is uncontrollable.

Just recently, Sun Group in Brown Univ. has developed iron oxide nanoparticle contrast agents with a hydrodynamic size of 8.4 nm and reported the MR contrast effect. The report has revealed that the contrast effect increased for tumor tissues, but a much higher contrast effect was observed in the spleen because of more than several times of nanoparticles being delivered to the liver and the spleen (Xie, J. et al, Journal of the American Chemical Society, 2008, 130, 7542-7543). In their case, it is considered that more nanoparticles are delivered to the liver because a high density of penyl-group on the surface of the nanoparticle badly affects biocompatibility.

This research group has also developed iron oxide contrast agents or quantum dot contrast agents, which are 10 nm in hydrodynamic size, bound to targeting molecules and biocompatible molecules, and have water-solubilities. Even in this case, it has been observed from an animal experiment that very excellent selectivities was exhibited within marker-expressed cells, but the delivery ratios to the liver and the spleen were 4 times and 12 times higher than that to tumor tissues. Here, the number of biocompatible polymers (PEG) bound to the nanoparticles are limited to about 5 to 10 for each nanoparticle. Thus, it is determined that there is a limitation in exhibiting a biocompatible property.

Therefore, it is required to develop targeting bio-imaging nanoparticle contrast agents, capable of being effectively delivered more to tumor tissues other than the liver by reducing a ratio of being delivered to the liver and the spleen due to being seized by an immune system, if possible, by optimizing a hydrodynamic size less than 10 nm, dispersibility, bonding to target molecules and biocompatibility.

### SUMMARY OF THE INVENTION

Therefore, to address the requirements in this field, an aspect of the detailed description is to provide bio-imaging nanoparticles capable of exhibiting high contrast effect for tumor tissues by being more effectively delivered to the tumor tissues than to the liver and the spleen while migrating along the blood streams, without being seized by an immune system, by virtue of properties of a hydrodynamic size less than 10 nm, high water-solubility, bonding to targeting molecules and excellent biocompatibility.

To achieve these and other advantages and in accordance with the purpose of this specification, as embodied and broadly described herein, there is provided bio-imaging nanoparticles each comprising a core nanoparticle, a bonding layer having organic ligands, surfactants and polyoxyalkylene derivatives of fatty acid ester, and veiling the core nanoparticle, and functional molecules, wherein the organic ligands are bound to a surface of the core nanoparticle, the surfactants are bound to a portion of the surface of the core nanoparticle to which the organic ligands are not bound, the polyoxyalkylene derivatives of the fatty acid ester are introduced in an empty space between the organic ligands and the surfactants of the bonding layer, and the functional molecule is bound to a second terminal end opposite to a first terminal end in both terminal ends of the organic ligand, the first terminal end of the organic ligand being bound to a surface of the core nanoparticle.

A method for producing bio-imaging nanoparticles according to the exemplary embodiment of this specification may include (a) preparing core nanoparticles whose outer surface are coated with surfactants, (b) partially replacing the surfactants with organic ligands such that the organic ligands are bound on the surface of the core nanoparticles, (c) bonding functional molecules to second terminal ends, opposite to first terminal ends, of both terminal ends of the organic ligands, the first terminal end being bound to the surface of the core nanoparticle, and (d) introducing polyoxyalkylene derivatives of fatty acid ester between the organic ligands and the surfactants.

### Effect of the Invention

In accordance with the present disclosure, an excessive amount of polyoxyalkylene derivatives of fatty acid ester (Tween molecules) may be introduced between ligands of a hydrophobic targeting nanoparticle contrast agent, so as to obtain nanoparticles, as final resultants, each having a hydrodynamic size less than 10 nm, water-solubility, targetability and excellent biocompatibility, thereby being usable as contrast agents. As analysis results of tumor animal imaging and biodistribution using the nanoparticles, similar distribution has been observed in a quantity of iron delivered to tumor tissues and a quantity of iron delivered to the liver. This distribution exhibits the best lesion selectivity among biodistributions reported so far. This result can be usefully employed in an early diagnosis of diseases.

Further scope of applicability of the present application will become more apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from the detailed description.

### BRIEF DESCRIPTION OF THE DRAWINGS

The accompanying drawings, which are included to provide a further understanding of the invention and are incorporated in and constitute a part of this specification, illustrate exemplary embodiments and together with the description serve to explain the principles of the invention.

In the drawings:

FIG. 1 is a diagram of a contrast agent nanoparticle in accordance with the present disclosure;

FIG. 2 is a graph showing hydrodynamic size distributions of contrast agent nanoparticles fabricated in accordance with Example 1;

FIG. 3 is a TEM image of the contrast agent nanoparticles fabricated in accordance with Example 1;

FIG. 4 is a TEM image of contrast agent nanoparticles fabricated in accordance with Example 2;

FIG. 5 shows MRI images obtained from tumor animal models, to which the contrast agents fabricated in Examples 1 and 2 are injected;

FIG. 6 is a graph showing bio-distributions obtained from tumor animal models using the contrast agents fabricated in accordance with Examples 1 and 2; and

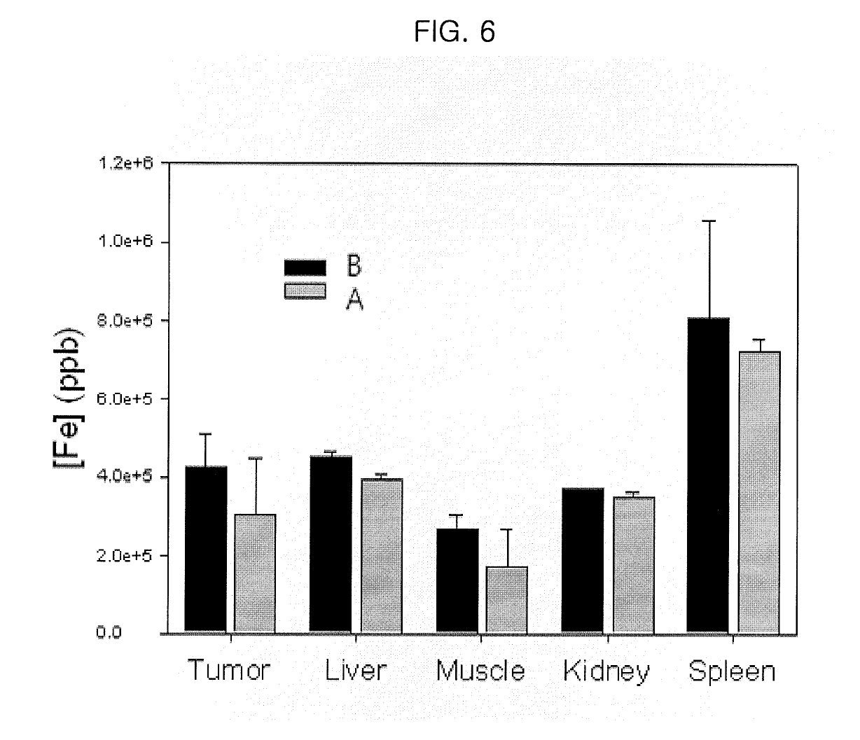

FIG. 7 is a diagram of a superparamagnetic cluster-nanoparticle-porous composite bead as one example of various core nanoparticles.

### EXPLANATION FOR THE MAJOR REFERENCE NUMERALS

1: superparamagnetic cluster (cluster composed of superparamagnetic nanoparticles)
2: porous bead
2-1: central porous bead
2-2: porous layer
3: nanoparticle
S: concentric sphere
10: core of core nanoparticle
12: shell of core nanoparticle
14: surfactant
16: polyoxyalkylene derivatives of the fatty acid ester
18: functional molecule
19: organic ligand

### DETAILED DESCRIPTION OF THE INVENTION

A bio-imaging nanoparticle according to the present disclosure may include a core nanoparticle, a bonding layer having organic ligands, surfactants and polyoxyalkylene derivatives of fatty acid ester, and veiling the core nanoparticle, and functional molecules, wherein the organic ligands are bound to a surface of the core nanoparticle, the surfactants are bound to a portion of the surface of the core nanoparticle to which the organic ligands are not bound, the polyoxyalkylene derivatives of the fatty acid ester are introduced in an empty space between the organic ligands and the surfactants of the bonding layer, and the functional molecule is bound to a second terminal end opposite to a first terminal end of both terminal ends of the organic ligand, the first terminal end of the organic ligand being bound to a shell of the core nanoparticle. With the configuration of the bio-imaging nanoparticles, namely, an excessive amount of polyoxyalkylene derivatives of fatty acid ester (Tween molecules) may be introduced between ligands of hydrophobic targeting nanoparticle contrast agents, so as to obtain nanoparticles, as final resultants, each having properties of a hydrodynamic size less than 10 nm, water-solubility, targetability and excellent biocompatibility, thereby being usable as contrast agents. As analysis results of tumor animal imaging and biodistribution using the nanoparticles, similar distribution has been observed in a quantity of iron delivered to tumor tissues and a quantity of iron delivered to the liver, unlike the related art bio-imaging nanoparticles.

The core nanoparticle may consist of core and shell, the core may be made of iron oxide, and the shell may be made of iron. Here, the present disclosure may not be limited to the structure. The core nanoparticle may include a superparamagnetic cluster, a central porous bead veiling the cluster, minute nanoparticles radially bonding to an outer surface of the core porous bead by an electrostatic attraction, and a porous layer formed to veil the minute nanoparticles, wherein the minute nanoparticles may be at least one selected from a group consisting of light-emitting nanoparticles, superparamagnetic nanoparticles, metallic nanoparticles and metal oxide nanoparticles, and the core nanoparticle may be various types of nanoparticles, such as superparamagnetic cluster-nanoparticles-porous composite bead.

The organic ligand may be prepared by connecting 1 to 30 organic ligands each having a hydrocarbon chain with 8 to 20 carbon atoms. A first terminal end of the organic ligand may be a thiol group, which may form a metal-thiolate bond with the surface of the core nanoparticle, and the second terminal end may be hydrophilic.

As one of main concepts of the present disclosure, the polyoxyalkylene derivative of fatty acid ester may be a branched type. The fatty acid ester may contain carbon atoms. The number of carbon atoms may be the same as the number of carbon atoms contained in the organic ligand or the difference therebetween may be 1 to 3 carbon atoms. In detail, the polyoxyalkylene derivative of fatty acid ester may be Tween 20, Tween 40, Tween 60 or Tween 80.

The functional molecule may be a biocompatible molecule, targeting molecule, a compound or a mixture thereof, as needed.

Meanwhile, the bonding layer containing the surfactants, the organic ligands and the polyoxyalkylene derivatives of fatty acid ester may veil the outer surface of the core nanoparticle, and the functional molecules may externally protrude from the bonding layer.

With regard to the coupling between the components, the organic ligands and the functional molecules may be coupled to each other by an amide bond or an ester bond. The organic ligands, the surfactants and the polyoxyalkylene derivatives of fatty acid ester may be coupled by the van der Waals force.

The bio-imaging nanoparticle may be partially hydrophilic due to containing the hydrophobic core nanoparticle and the hydrophilic organic ligands. That is, the bio-imaging nanoparticle has the hydrophobicity as a whole, but the hydrophilic organic ligand-coupled portion partially has the hydrophilicity.

A hydrodynamic size of the bio-imaging nanoparticle may be 10 nm or less.

A method for fabricating bio-imaging nanoparticles according to the present disclosure may include (a) preparing core nanoparticles whose outer surface is coated with surfactants, (b) partially replacing the surfactants with organic ligands such that the organic ligands are bound on the surfaces of the core nanoparticles, (c) bonding functional molecules to second terminal ends, opposite to first terminal ends in both terminal ends of the organic ligands, the first terminal ends being coupled to the surfaces of the organic nanoparticles, and (d) introducing polyoxyalkylene derivatives of fatty acid ester between the organic ligands and the surfactants.

In step (b), 1 to 30 molar equivalents of an organic ligand, which has both terminal ends with a thiol group and a hydrophilic group connected to each other by a hydrocarbon chain of 8 to 20 carbon atoms, may be added so as to form a metal-thiolate bond between the core nanoparticle and the first terminal end of the organic ligand having the thiol group. The polyoxyalkylene derivatives of fatty acid ester may be a branched type, and the fatty acid ester may contain carbon atoms. The number of carbon atoms of the fatty acid ester may be the same as the number of carbon atoms of the organic ligand, or a difference therebetween may be 1 to 3 carbon atoms.

In the method for fabricating the bio-imaging nanoparticles, the bonding layer containing the surfactants, the organic ligands and the polyoxyalkylene derivatives of fatty acid ester may veil the outer surface of the core nanoparticle. The functional molecule may externally protrude from the bonding layer. The core nanoparticle may be composed of core and shell. The core may be made of iron oxide and the shell may be made of iron. The organic ligands and the functional molecules may be coupled to each other by an amide bond or an ester bond. The organic ligands, the surfactants and the polyoxyalkylene derivatives of fatty acid ester may be coupled by the van der Waals force. The bio-imaging nanoparticle may be partially hydrophilic due to containing the hydrophobic core nanoparticle and the hydrophilic organic ligands. A hydrodynamic size of the bio-imaging nanoparticle may be 10 nm or less.

### EXAMPLES

Hereinafter, the present disclosure will be described in more detail according to Examples. Here, the examples are merely illustrative and may not be construed to limit the present disclosure.

### Example 1

### Fabrication of Nanoparticle Contrast Agent (SPION(-OA.Tw80)ₑₓ(-MHA-enPEG-cRGDfK)₁₀)

According to a well-known method (WOO, Kyeong-Ja et al 1, Method for the Production of Bio-Imaging Nanoparticles with High Yield by Early Introduction of Irregular Structure: Korean Patent No. 10-0943839, 20100217), hydrophobic targeting nanoparticle contrast agents SPION(-OA)ₑₓ(-MHA-enPEG-cRGDfK)₁₀ were fabricated. 20 ml of nanoparticles SPION(-OA)ₑₓ (7.4 nm) in an iron oxide/iron (core/shell) structure that a surface of iron oxide nanoparticle was covered with a shell made of iron was fabricated. To 6 mL of this resulting undiluted solution was added ethanol, and the resulting solution was centrifuged. The centrifuged solution was sequentially mixed with acetonitrile, centrifuged, purified and dispersed in 60 mL of toluene (Solution I, about 3×10⁻⁷ M nanoparticles). Meanwhile, a solution (MHA Solution) in which 1.7 mg of mercaptohexadecanoic acid (MHA) was dissolved in 10 mL of toluene and a solution (DCC Solution) in which 3.3 mg of dicyclohexylcarbodiimide (DCC) was dissolved in 10 mL of toluene were prepared. When the solution I was heated up to 100° C., 0.30 mL of the MHA solution (10 molar equivalents for each particle) was added to the solution I and stirred for 1 hour (3 mL of the solution was used for analysis). This solution was cooled using a water bath. When the solution was cooled down to 30° C., 0.33 mL of the DCC solution (30 molar equivalents for each particle) was added and stirred for 1 hour. To this mixture was added 1.05 mL of enPEG-cRGDfK solution (4.9×10⁻⁴ M in dimethylsulfoxide, 30 molar equivalents for each particle) and the solution was stirred for 20 hours. Here, the enPEG-cRGDfK is a material in which cyclic RGDfK as a peptide unit is connected to one end of polyethylene glycol diacid having a molecular weight of 600 and ethylenediamine is connected to the other end thereof by an amide bond. After 16 hours, as the reaction proceeded, the solution became a light suspension. The suspension was subject to an ultrasonic treatment within 1 second in a bath once per 30 minutes so as to well-disperse the particles and complete the reaction. Finally, the suspension was poured in a centrifugal container to be centrifuged. Thereafter, the solid was dispersed in 17.1 mL of chloroform, thereby fabricating is hydrophobic targeting nanoparticles SPION(-OA)ₑₓ(-MHA-enPEG-cRGDfK)₁₀.

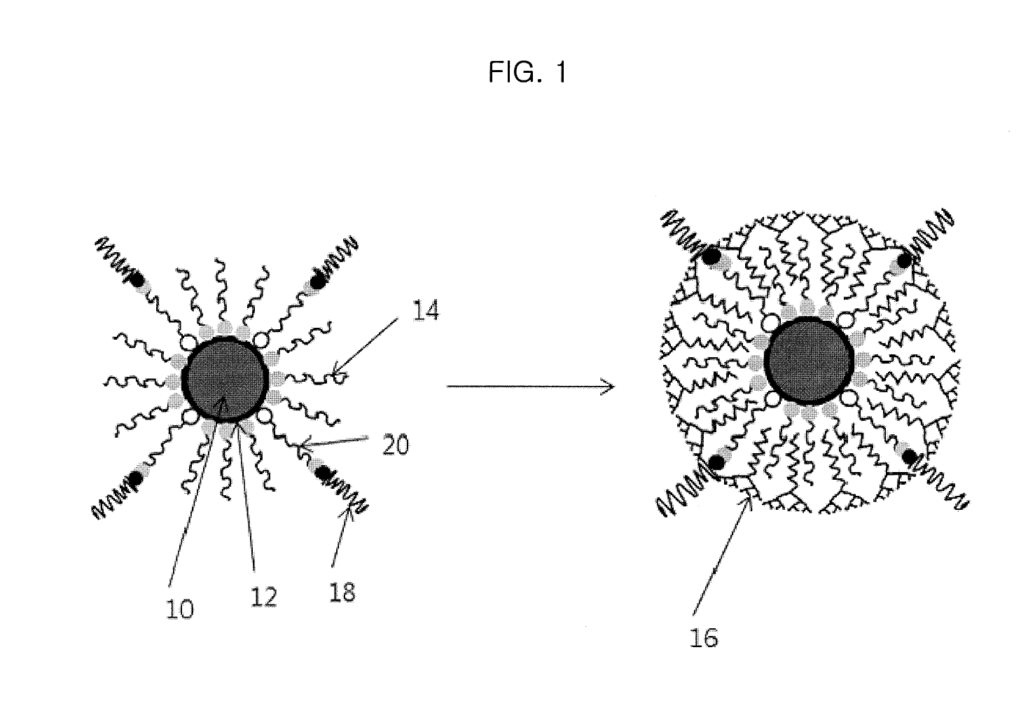

Afterwards, to the hydrophobic targeting nanoparticle SPION(-OA)ₑₓ(-MHA-enPEG-cRGDfK)₁₀ solution was added 0.63 mL of Tween 80 (32,000 molar equivalents for each particle) and weakly vortexed for 4 days such that Tween 80 molecules, which had oleic acid (OA) fatty acid as a part of molecules, could be introduced between OA surfactants and organic ligands, which already existed on the SPION surface. The bonding is made by van der Waals Force. This solution was connected to a vacuum pump to remove a solvent, and dispersed in distilled water (about 1×10⁻⁶ M). This dispersion was put in a dialysis jar (dialysis bag) and dialyzed for 1 day by placing in a beaker containing 1 L of distilled water for removal of surplus Tween 80 remaining in the solution. The nanoparticle solution contained in the jar was collected. The collected nanoparticle solution was kept in a vial as an undiluted solution (about 8×10⁻⁷ M, 20 mL) of SPION(-OA.Tw80)ₑₓ(-MHA-enPEG-cRGDfK)₁₀, which was the right structured material of FIG. 1, and used whenever needed.

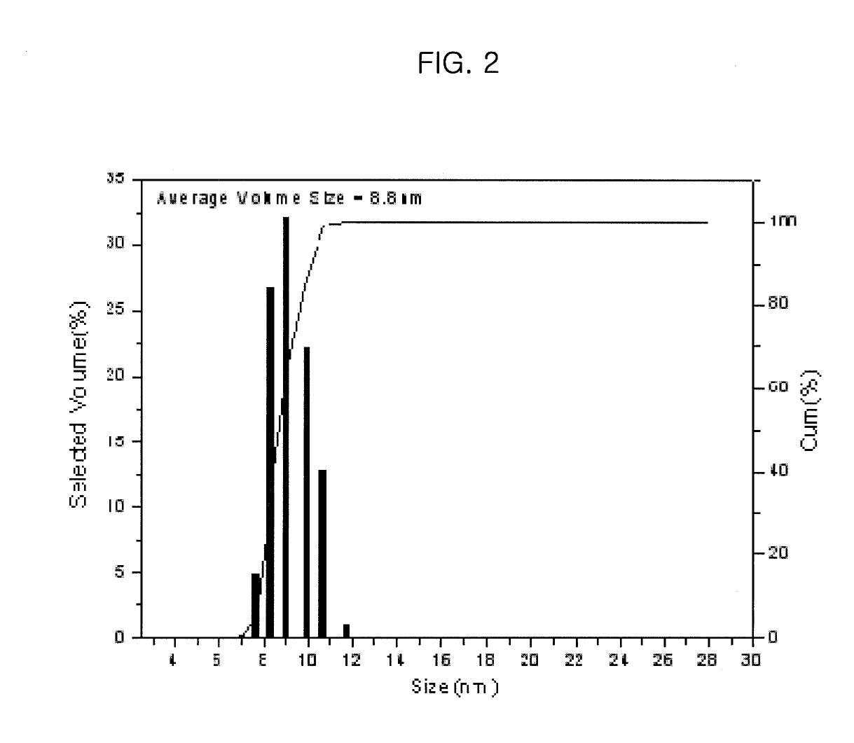

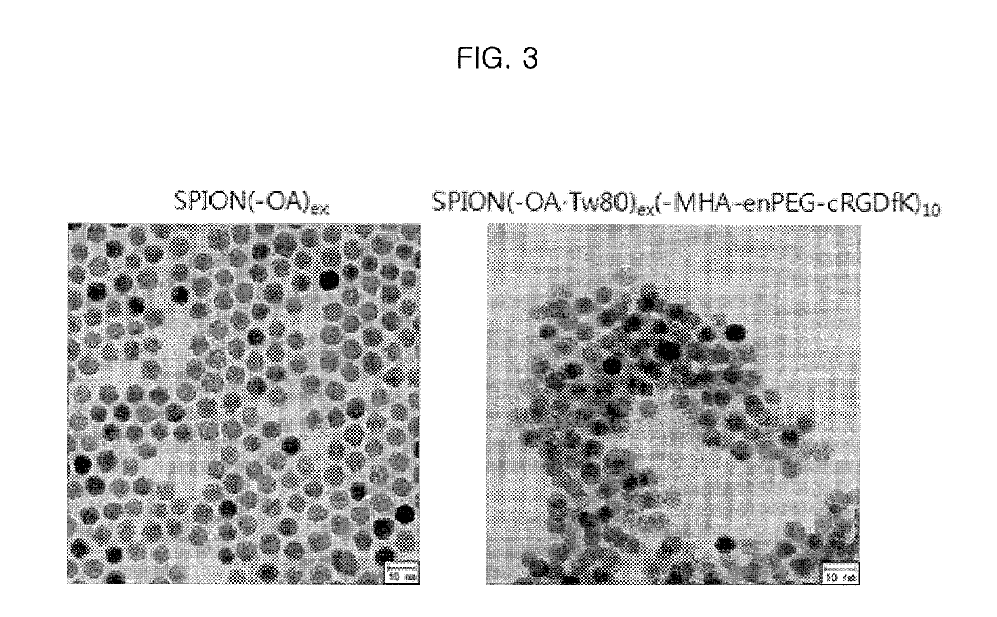

To check properties of the fabricated particles, a small amount of the solution was taken and requested from Korea Basic Science Institute to measure hydrodynamic sizes thereof. It was confirmed from the measurements that the particles were 8.8 nm in size and well dispersed without aggregation (FIG. 2). A TEM image of the undiluted solution of the finally obtained contrast agents to SPION(-OA-Tw80)ₑₓ(-MHA-enPEG-cRGDfK)₁₀ and that of the starting material SPION(-OA)ₑₓ were compared. The comparison results are shown in FIG. 3. It can be seen in the TEM image that the nanoparticles are separated individually. Also, the starting material shows that SPION particles form a two-dimensional self-assembly under the influence of hydrophobic OA molecules, whereas the final solution exhibits that the particles are overlapped under the influence of the Tween 80 molecules. FT-IR spectrum analysis was allowed to confirm the presence of all of peaks of iron oxide, cRGDfK, Tween 80 and the like, showing Fe—O band (˜602 cm⁻¹), C—O stretching band (1108 cm⁻¹), amide C═O band (1656 cm⁻¹ from cRGDfK), carboxy C═O band (1735 cm⁻¹, from Tween 80), C—H stretching band (˜2900 cm⁻¹), broad O—H band (˜3400 cm⁻¹) and the like. Consequently, the fabrication of a desired SPION(-OA.Tw80)ₑₓ(-MHA-enPEG-cRGDfK)₁₀ particles was indirectly confirmed.

### Example 2

### Fabrication of Nanoparticle SPION(-OA.Tw80)ₑₓ

To execute a comparative experiment for targetability of the nanoparticle contrast agents synthesized in Example 1, SPION(-OA.Tw80)ₑₓ which had the same properties to the particles of Example 1 except for the targetability were fabricated as follows.

20 ml of nanoparticles SPION(-OA)ₑₓ (7.4 nm) in an iron oxide/iron core/shell structure that a surface of iron oxide nanoparticle was covered with a shell made of iron was fabricated. To 6 mL of this resulting undiluted solution was added ethanol, and the resulting solution was centrifuged. The centrifuged solution was sequentially mixed with acetonitrile, centrifuged, purified and dispersed in 18 mL of toluene (Solution II, about 1×10⁻⁶ M nanoparticles). To the solution II was added 0.63 mL of Tween 80 (32,000 molar equivalents for each particle) and weakly vortexed for 4 days to introduce Tween 80 molecules between OA(Oleic Acid) ligands. This solution was connected to a vacuum pump to remove a solvent and dispersed in distilled water (about 1×10⁻⁶ M, 18 mL). The dispersion was dialyzed for 1 day using 1 L of distilled water for removal of surplus Tween 80 remaining in the solution. The nanoparticle solution was collected. The collected nanoparticle solution was kept in a vial as an undiluted solution (about 7.8×10⁻⁷ M, 23 mL) of SPION(-OA.Tw80)ₑₓ, and used whenever needed.

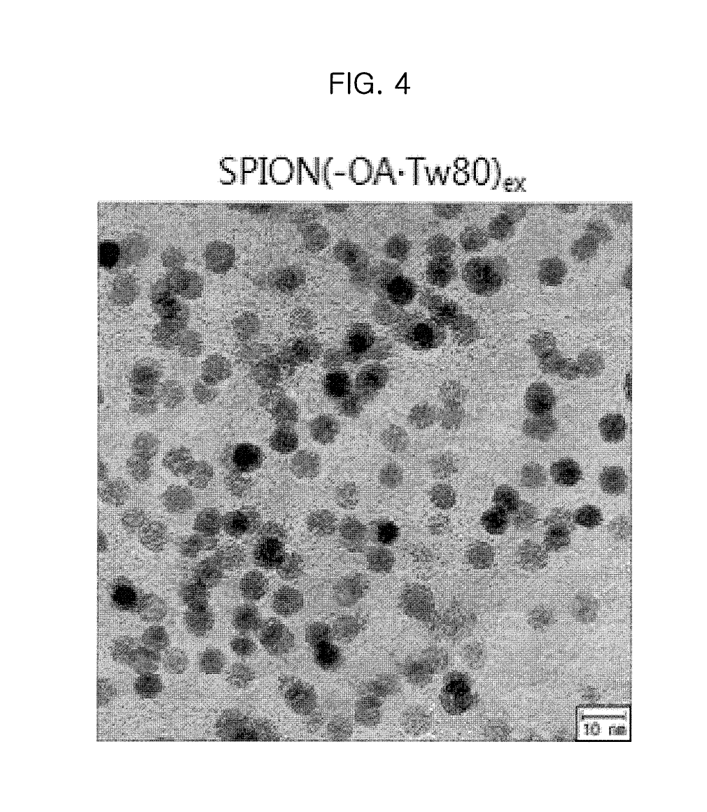

This solution was sent to KIST Advanced Analysis Center to analyze a content of iron according to an AAS method. The analysis results showed that the iron concentration of the undiluted solution was 249 ppm. Accordingly, a particle concentration was calculated by considering a particle size of 7.4 nm obtained by a TEM image and a particle density of 4.856 g/cm³. The calculated particle concentration was 5.73×10⁻⁷ M, whereby it was confirmed that the particle concentration was not so different from an estimated concentration of 7.8×10⁻⁷ M. Also, a small amount of the solution was taken and requested from Korea Basic Science Institute to measure hydrodynamic sizes thereof. It was confirmed from the measurements that the particles were 8.2 nm in size and well dispersed without aggregation. A TEM image of the thusly-obtained undiluted solution SPION(-OA.Tw80)ₑₓ was shown in FIG. 4.

### Example 3

### Tumor Animal Experiment

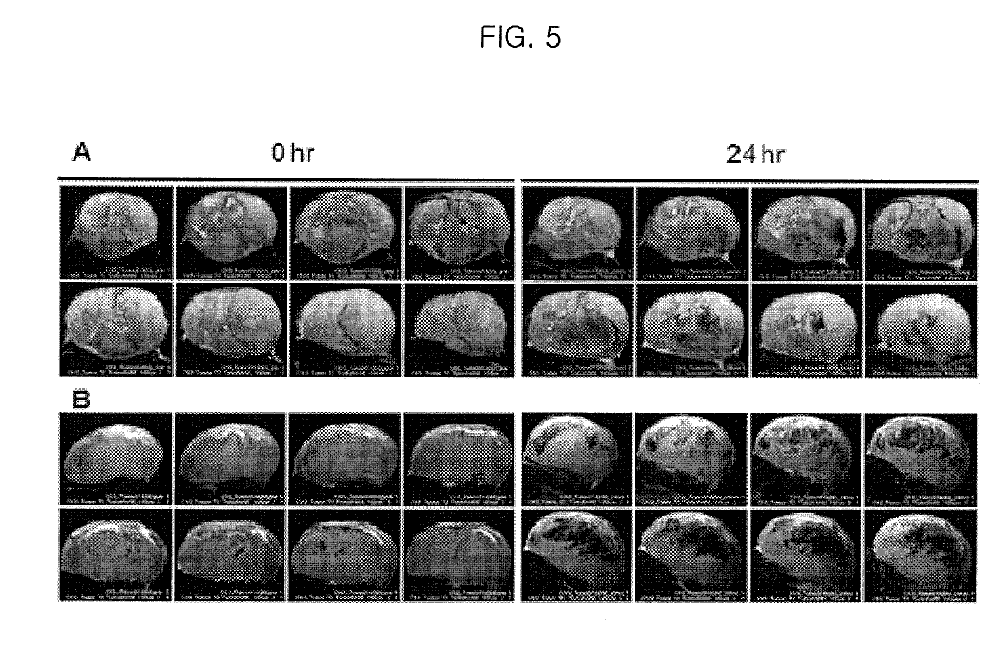

CT-26 colon carcinoma cell line was hypodermically injected into mice, thereby preparing a pair of tumor animal models for each condition. When tumor tissues were grown to 10 mm, the contrast agents synthesized in Examples 1 and 2 were injected into those mice, respectively. The tumor animal models were sacrificed before the injection of the contrast agents and when 24 hours elapsed after the injection of the contrast agents, respectively, thereby obtaining MRIs of tumor tissues, which are shown in FIG. 4. As shown in FIG. 4, the contrast agents synthesized in Examples 1 and 2 all exhibited an increased shade after the injection rather than before the injection. Also, SPION(-OA.Tw80)ₑₓ(-MHA-enPEG-cRGDfK)₁₀ indicated with B exhibited much more increased shade than SPION(-OA.Tw80)ₑₓ indicated with A. In addition, ICP-MS analysis was carried out for iron ingredients for each region of the tumor animal models so as to examine biodistribution of the targeting contrast agents, which are shown in FIG. 5. The iron ingredients by the contrast agent were detected from tumor tissues and the liver, and two times more than those from the tumor tissues and the liver were detected from the spleen. Accordingly, it was confirmed that this contrast agent was delivered to the tumor tissues at a remarkably higher ratio than that of any contrast agent which has been reported so far. For reference, it was noticed in a contrast agent, whose surface was modified to be water-soluble by using mercaptopropionic acid instead of Tween 80 at the last step of Example 1, that a delivery ratio to the liver and a deliver ratio to the spleen were 4 times and 12 times higher than that to the tumor tissues.

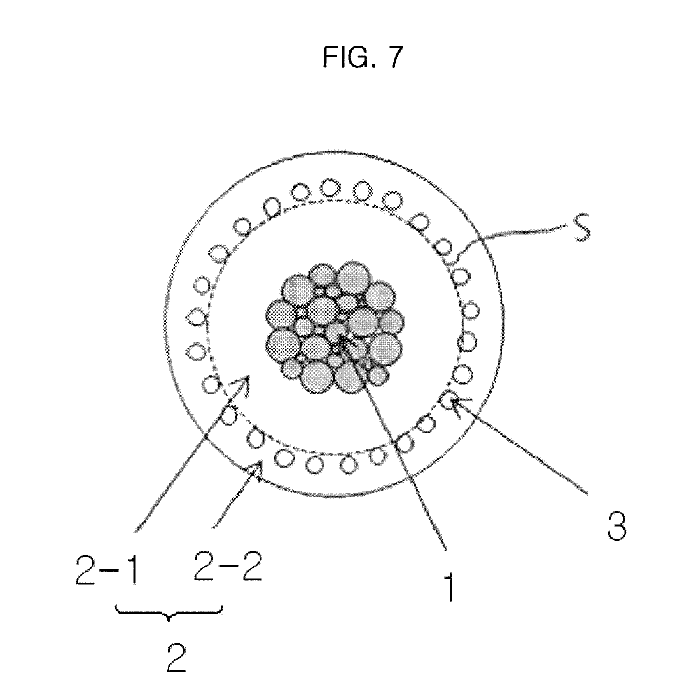

Meanwhile, instead of the core nanoparticle of Example 1 may be used a nanoparticle, as shown in FIG. 7, which includes a superparamagnetic cluster, a central porous bead veiling the cluster, minute nanoparticles radially bonding to an outer surface of the core porous bead by an electrostatic attraction, and a porous layer formed to veil the minute nanoparticles. Here, the minute nanoparticles may be at least one selected from a group consisting of light-emitting nanoparticles, superparamagnetic nanoparticles, metallic nanoparticles and metal oxide nanoparticles. Nanoparticle to be used instead of the core nanoparticle may be superparamagnetic cluster-nanoparticles-porous composite bead. The succeeding processes may be performed as the same as Example 1 to fabricate various types of nanoparticles.

The foregoing embodiments and advantages are merely exemplary and are not to be construed as limiting the present disclosure. The present teachings can be readily applied to other types of apparatuses. This description is intended to be illustrative, and not to limit the scope of the claims. Many alternatives, modifications, and variations will be apparent to those skilled in the art. The features, structures, methods, and other characteristics of the exemplary embodiments described herein may be combined in various ways to obtain additional and/or alternative exemplary embodiments.

As the present features may be embodied in several forms without departing from the characteristics thereof, it should also be understood that the above-described embodiments are not limited by any of the details of the foregoing description, unless otherwise specified, but rather should be construed broadly within its scope as defined in the appended claims, and therefore all changes and modifications that fall within the metes and bounds of the claims, or equivalents of such metes and bounds are therefore intended to be embraced by the appended claims.

What is claimed is:

1. A bio-imaging nanoparticle comprising:
a core nanoparticle;
a bonding layer having organic ligands, surfactants and polyoxyalkylene derivatives of fatty acid ester, the bonding layer veiling the core nanoparticle; and
functional molecules,
wherein the organic ligands are bound to a surface of the core nanoparticle,
wherein the surfactants are bound to a portion of the surface of the core nanoparticle to which the organic ligands are not bound,
wherein the polyoxyalkylene derivatives of the fatty acid ester are introduced in an empty space between the organic ligands and the surfactants of the bonding layer, and
wherein the functional molecule is bound to a second terminal end opposite to a first terminal end in both terminal ends of the organic ligand, the first terminal end of the organic ligand being bound to a surface of the core nanoparticle.

2. The nanoparticle of claim 1, wherein the core nanoparticle consists of core and shell, the core is made of iron oxide, and the shell is made of iron.

3. The nanoparticle of claim 1, wherein the organic ligand is a molecule which has a hydrocarbon chain with 8 to 20 carbon atoms and the number of organic ligands connected to the core nanoparticle is 1 to 30.
wherein the first terminal end of the organic ligand is a thiol group, the thiol group forming a metal-thiolate bond with the core nanoparticle, and
wherein the second terminal end of the organic ligand is hydrophilic.

4. The nanoparticle of claim 1, wherein the polyoxyalkylene derivative of fatty acid ester is a branched type,
wherein the fatty acid ester contains carbon atoms, and the number of carbon atoms is the same as the number of carbon atoms contained in the organic ligand or has a difference there between being 1 to 3 carbon atoms.

5. The nanoparticle of claim 1, wherein the polyoxyalkylene derivative of fatty acid ester is Tween 20, Tween 40, Tween 60 or Tween 80.

6. The nanoparticle of claim 1, wherein the functional molecule is a biocompatible molecule, targeting molecule, a composite or a mixture thereof.

7. The nanoparticle of claim 1, wherein the bonding layer containing the surfactants, the organic ligands and the polyoxyalkylene derivatives of fatty acid ester veils the outer surface of the core nanoparticle, and the functional molecules externally protrudes from the bonding layer.

8. The nanoparticle of claim 1, wherein the organic ligands and the functional molecules are bound to each other by an amide bond or an ester bond.

9. The nanoparticle of claim 1, wherein the organic ligands, the surfactants and the polyoxyalkylene derivatives of fatty acid ester are coupled by the van der Waals force.

10. The nanoparticle of claim 1, wherein a hydrodynamic size of the bio-imaging nanoparticle is 10 nm or less.

11. A method for fabricating bio-imaging nanoparticles comprising:
(a) preparing core nanoparticles whose outer surface is coated with surfactants;
(b) partially replacing the surfactants with organic ligands such that the organic ligands are bound on the surface of the core nanoparticles;
(c) bonding functional molecules to second terminal ends, which is opposite to first terminal ends, of both terminal ends of the organic ligands, and the first terminal end is bound to the surface of the core nanoparticle; and
(d) introducing polyoxyalkylene derivatives of fatty acid ester between the organic ligands and the surfactants.

12. The method of claim 11, wherein in step (b), 1 to 30 equivalents of an organic ligand, is added to form a metal-thiolate bond between the core nanoparticle and the first terminal end of the organic ligand having the thiol group, the organic ligand having both terminal ends with the thiol group and a hydrophilic group connected to each other by a hydrocarbon chain of 8 to 20 carbon atoms.

13. The method of claim 11, wherein the core nanoparticle consists of core and shell, the core is made of iron oxide, and the shell is made of iron.

14. The method of claim 11, wherein the polyoxyalkylene derivative of fatty acid ester is a branched type,
wherein the fatty acid ester contains carbon atoms, and the number of carbon atoms is the same as the number of carbon atoms contained in the organic ligand or has a difference therebetween being 1 to 3 carbon atoms.

15. The method of claim 11, wherein the polyoxyalkylene derivative of fatty acid ester is Tween 20, Tween 40, Tween 60 or Tween 80.

16. The method of claim 11, wherein the functional molecule is a biocompatible molecule, targeting molecule, a composite or a mixture thereof.

17. The method of claim 11, wherein the bonding layer containing the surfactants, the organic ligands and the polyoxyalkylene derivatives of fatty acid ester veils the outer surface of the core nanoparticle, and the functional molecules externally protrudes from the bonding layer.

18. The method of claim 11, wherein the organic ligands and the functional molecules are bound to each other by an amide bond or an ester bond.

19. The method of claim 11, wherein the organic ligands, the surfactants and the polyoxyalkylene derivatives of fatty acid ester are coupled by the van der Waals force.
